(19) United States
(12) Patent Application Publication
(10) Pub. No.: US 2006/0281996 A1
(43) Pub. Date: Dec. 14, 2006

(54) Method of electrocardiogram (ECG) anaylysis and device thereof

(76) Inventors: Terry B.J. Kuo, Ji-an Township (TW); Jin-Jong Chen, Taipei City (TW); Wei-Fong Kao, Taipei City (TW); Chein-Chung Kuo, Hualien City (TW); Li-Yao Weng, Taipei City (TW)

Correspondence Address: J C PATENTS, INC., 4 VENTURE, SUITE 250, IRVINE, CA, 92618, US

(21) Appl. No.: 11/233,673

(22) Filed: Sep. 22, 2005

(30) Foreign Application Priority Data
Jun. 14, 2005 (TW) 94119592

(51) Int. Cl. A61B 5/04

(52) U.S. Cl. 600/509

(57) ABSTRACT

An analysis method of electrocardiogram (ECG) and the device thereof are provided. The method diagnoses a subject by non-invasive method to see whether the subject is at high risk of high mountain disease (HMD). The ECG analysis method comprises: first, detecting the ECG of the subject; transforming the ECG to obtain a plurality of heart rate variability (HRV) parameters; analyzing the HRV parameters and outputting an analyzed result.

### CROSS-REFERENCE TO RELATED APPLICATION

This application claims the priority benefit of Taiwan application serial no 94119592, filed on Jun. 14, 2005. All disclosure of the Taiwan application is incorporated herein by reference.

### BACKGROUND OF THE INVENTION

1. Field of Invention

The present invention relates to an analysis method of electrocardiogram (ECG) and the device thereof. More specifically, the present invention relates to diagnosing whether a subject is at the high-risk of high mountain disease (HMD) through ECG or electroencephalogram (EEG).

2. Description of Related Art

Mountainous areas in Taiwan with various and rich natural resources occupy about 70% of the whole island. In addition to the traditional forestry economic value, the mountainous areas further include the essential interests of ecological conservation, soil and water conservation and recreation activities. At present, in the advanced countries in the world, the potential of the priceless functions is highly valued. National parks and plural forest recreational areas provide people with good environment and space for various and particular activities. Fortunately, the unique mountainous landform and richness of forestry resources in Taiwan, and even the hills and parks extending into the urban areas all provide abundant and pleasant hiking environment. In mountainous areas, the temperature drops 0.6° C. in every 100 meter elevation of altitude; and every 300 meter elevation in altitude is equal to 480 kilometer movement towards the polar region. That is why the mountainous area is relatively cool even in the hottest summer. Besides the natural low-altitude tropical and subtropical landscapes, people in Taiwan can also enjoy the temperate and rigid forestry scenes of middle to high altitudes.

Mountainous areas above 2500 meter altitude and some restricted access forests are relatively inclement. Other than those for conservation purposes, the trails are seldom properly maintained and planned; they are usually rugged, rough and dangerous. Therefore, the wisdom and physical strength of people involved in mountain activities are challenged. As the altitude increases, the atmospheric pressure (especially the oxygen that people need during an activity) is drastically reduced that may be even riskier for one's physical strength. For example, on Yushan, the oxygen density on the top of the mountain is only 58% of the oxygen density at the sea level, thus may cause the symptoms of HMD such as headache, lethargy, dyspnea, nausea, vomiting, fatigue and blurred vision.

Generally, the physical strength will drop dramatically under anoxic condition. For altitude over 1,500 meters, the maximal oxygen uptake reduces by 1%-3.2% for every 305 meter elevation. The higher the altitude is, the more the body movement is affected. In particular, office workers without mountain activity training and lack of regular exercise will be restricted in their body movement, and even obvious discomfort symptom or acute mountain sickness (AMS) may occur. Many office workers having been busy with work for all weekdays usually rush to mountains excitedly at weekends. Without adequate and timely preparation, they normally do not have enough time to adapt themselves in such rapid elevation. As a result, injuries or accidents may occur because of sudden elevation and lack of oxygen once abruptly involved in mountain activities.

Altitude of HMD occurrence varies individually. The range can be very large. HMD may happen as low as 1828 meter altitude, or only as high as 4500 meters. According to the mountain climbers in Taiwan, the chance of HMD occurrences noticeably increases when the elevations are above 3,000 meters. Possible symptoms includes: headache, lethargy, hard to sleep, blurred vision, dyspnea, vomiting, anorexia, nausea, titubation, weakness, fatigue and abnormal happy feelings.

According to the statistics abroad, among all the non-trauma sicknesses happened during outdoor activities, HMD ranks the third just after cold and gastroenteritis. The symptoms of HMD include acute mountain sickness (AMS), high altitude pulmonary edema (HAPE) and high altitude cerebral edema (HACE); such illnesses occur due to low air pressure and anoxic circumstances. AMS usually occurs several hours or days after elevation (mostly happens within 24 hours). Generally speaking, the faster the elevation speed, the quicker the loss of energy, the weaker the individual physical condition, the more chance HMD likely to occur and the severer the sickness.

HMD is often misdiagnosed as flu, carsickness or pneumonia because of their similar symptoms. The symptoms include coughing, headache, dizziness, nausea, weakness, etc. and are possibly considered as flu. And the symptoms of headache, dizziness, nausea and vomiting are likely considered as carsickness on the mountainous roads. While the symptoms of the severer high altitude pulmonary edema (HAPE), such as coughing, dyspea, headache, dizziness, nausea, weakness, clear sputum or blood-streaked sputum are likely diagnosed as pneumonia. Incorrect diagnosis usually leads to incorrect treatment which delays the best opportunity of treatment. Therefore, besides the temporary use of Lake Louise AMS Questionnaire, the objective physiology index is helpful for accurate diagnosis.

### SUMMARY OF THE INVENTION

The object of the present invention is to provide a method of ECG analysis. The method can diagnose whether the subject is among the high risk of HMD according to ECG and the calculated heart rate variability (HRV) parameters of the subject.

Another object of the present invention is to provide an ECG analysis method. The ECG analysis method can diagnose whether the subject is among the high risk of HMD according to the EEG and the calculated EEG activity parameters of the subject.

Another object of the present invention is to provide an ECG analysis device which examines the heart rate of the subject with a noninvasive method. And through calculation and analysis, the analyzed result of whether the subject is among the high risk of HMD can be acquired.

Another object of the present invention is to provide an ECG analysis device which examines the brainwave of the subject with a noninvasive method. And through calculation and analysis, the analyzed result of whether the subject is among the high risk of HMD can be acquired.

The present invention provides an ECG analysis method which diagnoses the subject with a non-invasive method. The ECG analysis method includes: first, measure the heart rate of the subject to obtain ECG; secondly, convert the ECG to acquire a plurality of the HRV parameters; analyze based on the HRV parameters and output an analyzed result.

According to the embodiment of the present invention, the above HRV parameters include the variability parameter of low frequency density and the variability parameter of high frequency.

The present invention further provides an ECG analysis method. The ECG analysis method includes: first, the brainwave of the subject is measured to obtain EEG; secondly, the EEG is calculated to acquire a plurality of the EEG activity parameters; a plurality of the EEG activity parameters is analyzed against a plurality of the standard EEG activity values in the database; then the results are output.

According to the embodiment of the present invention, the above EEG activity parameters include EEG amplitude and EEG frequency.

The present invention further provides an ECG analysis device which diagnoses HMD of the subject with a non-invasive method. The analysis device includes a sensing unit, an analysis unit and an output unit. The sensing unit contacts with the skin surface of the subject's arm to sense and output the ECG of the subject. The sensing unit has a database. The ECG is magnified, filtered, digitized and transformally calculated to obtain a plurality of HRV parameters. Furthermore, the HRV parameters are analyzed and the pre-existing tables in the database are searched for the corresponding analyzed result. The output unit is responsible for receiving and outputting the analyzed result.

The present invention further provides an ECG analysis device including a sensing unit, an analysis unit and an output unit. The sensing unit contacts with the surface of the head of the subject, and is responsible for sensing and outputting the EEG of the subject. The analysis unit has a database. And the EEG is magnified, filtered, digitized and calculated by the analysis unit to acquire a plurality of EEG activity parameters. After the EEG activity parameters are analyzed, the pre-existing tables in the database are then searched for the corresponding analyzed result. The output unit is responsible for receiving and outputting the analyzed result.

Since the present invention uses non-invasive analysis device, it only takes five minutes to check whether the subject is among the high risk of HMD, and precautious measures can be taken before climbing mountains so as to avoid accident or being misdiagnosed as flu or pneumonia.

These and other exemplary embodiments, features, aspects, and advantages of the present invention will be described and become more apparent from the detailed description of exemplary embodiments when read in conjunction with accompanying drawings.

### BRIEF DESCRIPTION OF THE DRAWINGS

FIG. 1A is a schematic diagram of the ECG analysis device according to an embodiment of the present invention.

FIG. 1B is a schematic diagram of another ECG analysis device according to an embodiment of the present invention.

FIG. 1C schematically illustrates the side view of FIG. 1B according to an embodiment of the present invention.

FIG. 2A is a schematic diagram of an ECG analysis device according to an embodiment of the present invention.

FIG. 2B schematically illustrates the diagram of another ECG analysis device according to an embodiment of the present invention.

FIG. 3A to FIG. 3D respectively schematically illustrates the differences between the HRV parameters of HMD patients and the HRV parameters of non-HMD people.

FIG. 4A to FIG. 4D respectively schematically illustrates the differences between the EEG activity parameters of HMD patients and the EEG activity parameters of non-HMD people.

### DETAILED DESCRIPTION OF THE PREFERRED EMBODIMENTS

The ECG analysis method and the device thereof of the present invention mainly perform diagnosis using physical diagnosis technology. The so-called physical diagnosis technology generally refers to the medical diagnosis method of collecting EEG, ECG or the physical signals using instruments.

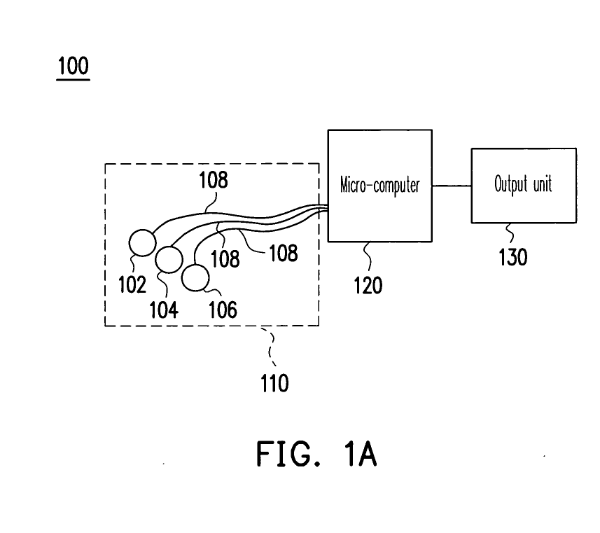

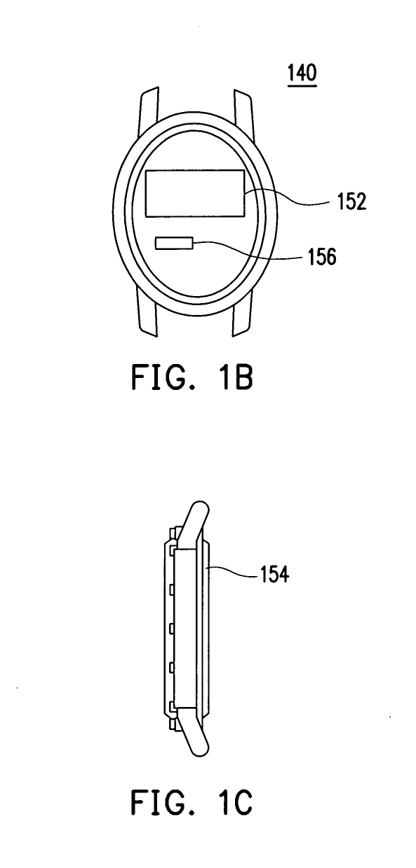

With reference to FIG. 1A, FIG. 1A is a schematic diagram of the HMD analysis device according to an embodiment of the present invention. The analysis device 100 includes a sensing unit 110, an analysis unit 120 and an output unit 130.

In the present embodiment, the sensing unit 100, for example, has a plurality of electrode pads 102, 104, 106 and a plurality of signal collecting wires 108. These electrode pads 102, 104, 106 stick on the surface of the arm skin of the subject to sense and output the ECG of the subject. Those skilled in the art can understand that these signal collecting wires 108, for example, can be button-like connectors of the electrode pads; one of the electrode pads can be stuck on the back end of the left hand while the other electrode pad can be stuck on the front end of the left hand of the subject, and another electrode pad can be stuck on the front end of the right hand (using the standard Lead I applying method). However, it is not limited to this method.

The analysis unit 120 has a database (not shown) which stores multiple forms of sorted diagnostic descriptions and tables for query. The analysis unit 120 receives ECG through these signal collecting wires 108. And the ECG is magnified, filtered, digitized and transformally calculated to acquire a plurality of HRV parameters. And it can be seen by people who are skilled in the art that the analysis unit 120 can include a first high-pass filter, a first magnifier, a first low-pass filter, a voltage current converter, a comparison circuit, a second high-pass filter, an optoisolator, an analog/digital converter and an RS-232 port. Nevertheless, it is not limited to the above description.

After the HRV parameters are received, the analysis unit 120 calculates, compares and analyzes these HRV parameters. Then, the tables in the database of the analysis unit 120 are searched for the corresponding analyzed result.

In the present invention, the output unit 130 is coupled to the analysis unit 120. The output unit 130 receives and outputs the analyzed result. Those skilled in the art can understand that the output unit 130 can be a monitor or a printer to display or print the examination report, or a CD burner to burn the examination report on a CD. Even, the subject can be examined by a nurse who will use the internet system as the output unit 130 to send the examination report to the remote terminal (for example, a doctor's analysis unit). However, it is not limited to the above description.

In the embodiment of the present invention, the analysis unit 120 is a computer with digital signal processing (DSP), and the computer can perform frequency-domain analysis, time-domain analysis and non-linear analysis.

With reference to FIG. 1B, FIG. 1B is a schematic diagram of another ECG analysis device according to an embodiment of the present invention. In the present embodiment, the analysis device 140, for example, can be a watch. The ECG of the subject is sensed by the sensing unit 154, and the ECG is calculated and analyzed by the micro-computer (not shown) in the watch, and the analyzed result is displayed on the output unit 152.

In the embodiment of the present invention, the button 156 can be, for example, an existing functional button on the watch.

In the present embodiment, the action principle of the analysis device 100 will be described in detail in the following ECG analysis method.

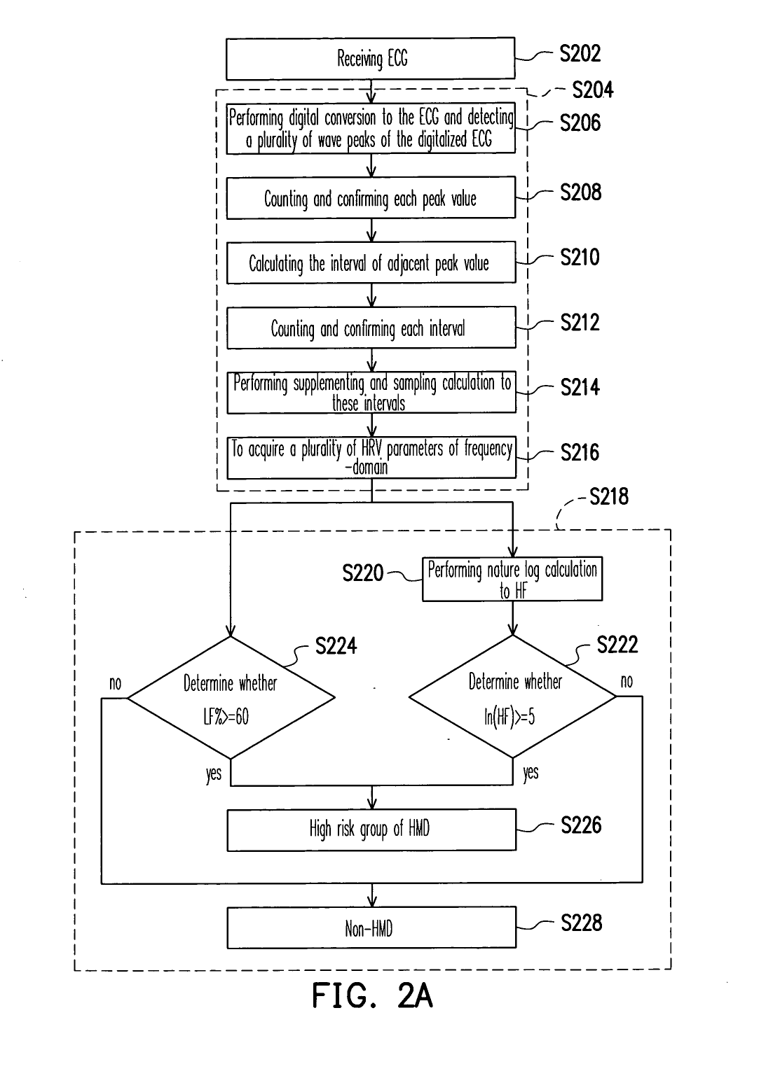

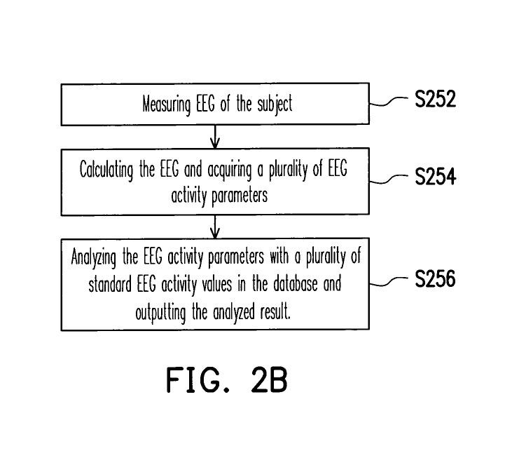

With reference to FIG. 2A, FIG. 2A is a schematic diagram of an ECG analysis device according to an embodiment of the present invention. In the present embodiment, the subject receives a five minute ECG collection.

First, the heart rate of the subject is measured to obtain ECG (S202). Next, the collected ECG is transformed to acquire a plurality of HRV parameters (S204). Wherein, step S204 includes transforming the ECG from time-domain into frequency-domain using Fast Fourier Transform; a plurality of HRV parameters including R—R interval, low frequency (LF) variability parameter, high frequency (HF) variability parameter, low frequency density (LF %) and low-frequency/high-frequency (LF/HF) variability parameters are acquired.

In the embodiment of the present invention, first the time point created by R wave is located through a peak detecting program, and the heart rate of each heartbeat can be calculated through the reciprocal of the time interval of the time point. And the time continuity thereof is maintained by a “sample and hold” procedure. The refresh rate of the “sample and hold” procedure is 16/second. The consecutive extended ECG is divided into groups, and each group (or referred to as an analysis window) is divided into 64 seconds (1024 points). In each of the analysis windows, the linear trend of the signal is eliminated first to avoid the low frequency interference. And the Hamming calculation is also used to avoid the interference between the individual frequency ingredients in the frequency spectrum. Next, the signal is then transformed into power frequency spectrum through the Fast Fourier Transform method.

Therefore, in each analysis, a 288 seconds heart rate at rest is collected. These data can be divided into 8 analysis windows, with each analysis window having 64 seconds (1024 points) length and overlapped by 50%. For each analysis window, the linear trend of the signal is eliminated first to avoid the low frequency interference. The Hamming calculation is also used to avoid the interference between the individual frequency ingredients in the frequency spectrum. Next, the signal is then transformed into frequency spectrum through the Fast Fourier Transform method. The frequency spectrum of 8 analysis windows is averaged to acquire the average periodogram (Kuo et al. 1999) whose frequency resolution can be up to 0.0167 ( 1/64) Hz. After the frequency spectrum of the 8 analysis windows is averaged, the stochastic noise can also be attenuated to signalize the frequency spectrum ingredient of high reproduction. In the present experiment, the 3 frequency spectrum elements include very low frequency (VLF, 0-0.04 Hz), low-frequency (LF, 0.04-0.15 Hz) and high-frequency (HF, 0.15-0.4 Hz) are quantitated using integral method. In the meantime, the quantitative parameters including total power (TP), low frequency/high frequency (LF/HF), low frequency density (LF %) and high frequency density (HF %) are calculated.

In the above step of S204, the received ECG is transformed to acquire a plurality of HRV parameters. The detailed procedure is: the ECG is transformed digitally first and a plurality of the digitalized ECG peaks is detected.

In the present embodiment, when the peak detection is completed, each peak is subjected to statistic and confirmative step (S208). Next, a plurality of the peak intervals of these peaks is calculated and acquired by the analysis unit, and each of these peak intervals is statistically counted and confirmed (S210). Herein, the analysis unit is used to calculate the intervals between peaks so as to acquire the intervals of a plurality of peaks. After these peak intervals are acquired, each of the peak intervals between these peaks is subjected to the action of statistic and confirmation (S212).

Lastly, the peak intervals are calculated by the analysis unit to acquire the frequency-domain of these HRV parameters. Herein, to calculate against the peak intervals is to perform supplementing and sampling calculation (S214) to these peak intervals, so as to acquire the frequency-domain of these HRV parameters (S216).

Next, analyze these HRV parameters and output an analyzed result (S218). Herein, the detailed procedure in S218 is to perform reciprocal calculation (S222) to the high frequency variability parameter in the HRV parameters to acquire In (HF). The value of In (HF) is then evaluated to see whether it is larger than or equal to 5 (S222). If the value of In (HF) is larger than or equal to 5, it can be determined that the subject is among the high risk of HMD and the analyzed result is output (S226). Otherwise, if the value of In (HF) is smaller than 5, it can be determined that the subject is not among the high risk of HMD, and the analyzed result is output (S228).

Besides the evaluation based on the high frequency variability parameter, the low frequency density (LF %) in the HRV parameters can also be used as the basis of evaluation; that is, to determine if the value of low frequency density is greater than or equal to 60 (S224). If the value of low frequency density is greater than or equal to 60 (S224), then it can tell that the subject is among the high risk of HMD, and the analyzed result (S226) is output. Contrarily, if the value of low frequency density is evaluated to be smaller than 60, it can tell that the subject is not among the high risk of HMD and the analyzed result (S228) is output.

In the present embodiment, as shown in the flow of FIG. 2B, the EEG can also be used to diagnose whether the subject is among the high risk of HMD. First, for example, the brainwave of the subject is measured using an EEG measuring instrument to obtain EEG (S252). Next, the EEG is calculated, and a plurality of activity parameters of EEG is acquired (S254). Herein, the calculation to the EEG, for example, can be magnification, filtering, digitization and transformation; however, it is not limited to these.

Next, the acquired parameters of EEG variability is evaluated against a plurality of standard EEG activity values (i.e. the EEG activity parameters of non-high risk of HMD), and the analyzed result is output (S256). Herein, output of the analyzed result is similar with steps S226 and S228, so it is not described in detail.

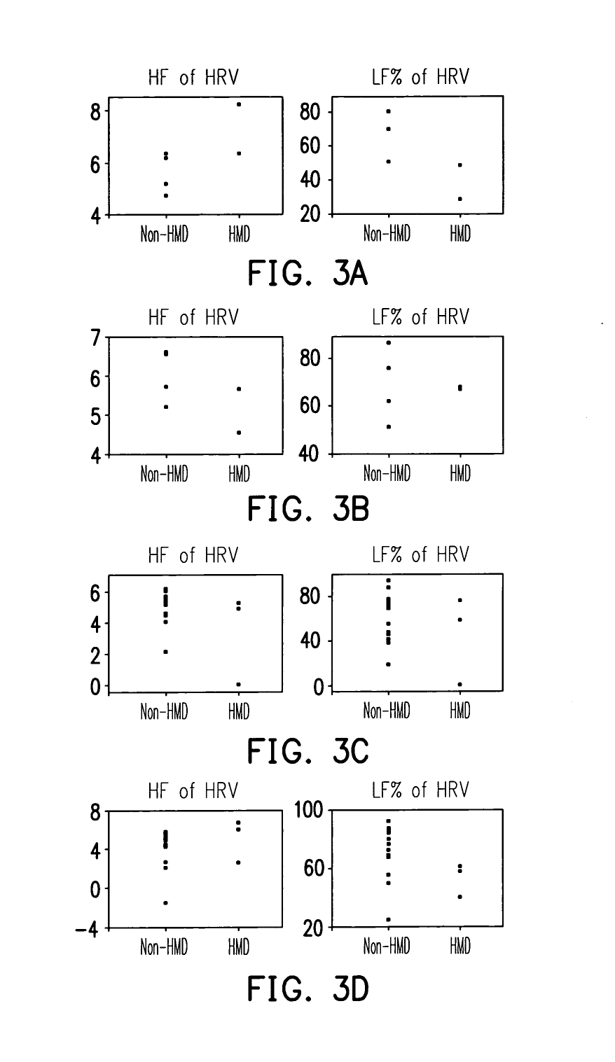

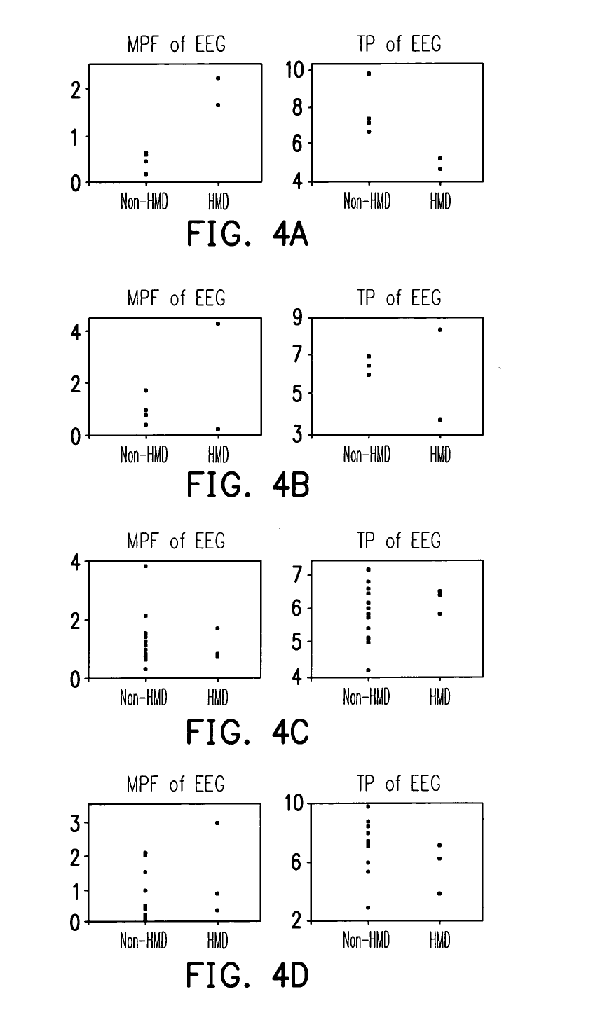

With reference to FIG. 3A to FIG. 3D and FIG. 4A to FIG. 4D, they respectively schematically illustrate differences between the HRV parameters of HMD patients and of normal people, and differences between the EEG activity parameters of HMD patients and of non-HMD people. The parameters are measured at different times according to an embodiment of the present invention. In here, FIG. 3A and FIG. 4A are the results measured on flat land; FIG. 3B and FIG. 4B are the results measured the day before mountain climbing; FIG. 3C and FIG. 4C are the results measured on the evening of the first day of mountain climbing; FIG. 3D and FIG. 4D are the results measured on the evening of the second day of mountain climbing.

In the present embodiment, Table 1 shows the comparison of the physiological values of the HMD patients and the non-HMD people measured when they are on the flat land.

TABLE 1

| | | No. of | | Std. | P |
|---|---|---|---|---|---|
| | | Testees | Mean | Deviation | Value |
| Red blood cell | Non-HMD | 15 | 4.243 | 0.802 | 0.175 |
| volume | HMD | 17 | 4.585 | 0.543 | |
| | HMD | | | | |
| Haemachrome | Non-HMD | 25 | 13.1280 | 0.9181 | 0.128 |
| | HMD | 20 | 13.6800 | 1.4577 | |
| Heart rate at rest | Non-HMD | 15 | 76.53 | 10.39 | 0.165 |
| | HMD | 15 | 81.40 | 8.14 | |
| Hemoglobin oxygen | Non-HMD | 15 | 98.27 | 1.28 | 0.567 |
| saturation | HMD | 16 | 98.06 | 0.57 | |
| Respiratory total | Non-HMD | 18 | −2.667 | 0.736 | 0.794 |
| amplitude | HMD | 18 | −2.744 | 1.017 | |
| Respiratory rate | Non-HMD | 18 | 0.25967 | 0.07169 | 0.182 |
| | HMD | 18 | 0.22711 | 0.07182 | |
| High frequency | Non-HMD | 18 | 5.0206 | 1.0568 | 0.024 |
| ingredient | HMD | 18 | 5.8783 | 1.1210 | |
| Low frequency | Non-HMD | 18 | 67.517 | 14.611 | 0.018 |
| density | HMD | 18 | 55.322 | 14.823 | |
| Low Frequency/ | Non-HMD | 18 | 1.1078 | 0.7688 | 0.012 |
| High Frequency | HMD | 18 | 0.4583 | 0.6989 | |
| EEG amplitude (TP) | Non-HMD | 18 | 6.2978 | 1.2664 | 0.289 |
| | HMD | 18 | 5.8989 | 0.9283 | |
| EEG frequency | Non-HMD | 18 | 4.68433 | 3.07490 | 0.888 |
| (MPF) | HMD | 18 | 4.55056 | 2.57399 | |

As shown in Table 1, in the aspect of blood ingredient analysis, there is no noticeable difference between the red blood cell volume and hemoglobin oxygen saturation of the HMD patients and of the non-HMD people. In the aspect of heart rates and hemoglobin oxygen saturation, the flat land rest heart rates of HMD patients are higher than that of non-HMD people, but there is no noticeable difference in the hemoglobin oxygen saturation. In the aspect of respiration depth and respiration rate, there is no noticeable difference between the HMD patients and the non-HMD people. In the aspect of ECG, both the heart rate at rest and the high frequency ingredient of the HMD patients on the flat land are higher than those of the non-HMD people. However, the low frequency density and low frequency/high frequency ingredient of the HMD patients are lower than those of the non-HMD people. In the aspect of EEG activity parameters, the EEG amplitude of the HMD patients is lower than that of the non-HMD people.

In the present embodiment, Table 2 shows the comparison of the physiological values of the HMD patients and non-HMD people measured when they are on the mountains.

TABLE 2

| | | No. of | | Std. | P |
|---|---|---|---|---|---|
| | | Testees | Mean | Deviation | Value |
| Heart rate at rest | Non-HMD | 14 | 96.07 | 14.83 | 0.338 |
| | HMD | 16 | 100.88 | 11.66 | |
| Hemoglobin oxygen | Non-HMD | 14 | 88.71 | 3.41 | 0.057 |
| saturation | HMD | 17 | 86.24 | 3.53 | |
| Respiratory total | Non-HMD | 23 | −1.956 | 1.094 | 0.240 |
| amplitude | HMD | 17 | −2.306 | 0.592 | |
| Respiratory rate | Non-HMD | 23 | 0.30465 | 0.062 | 0.663 |
| | HMD | 17 | 0.29424 | 0.71 | |
| High frequency | Non-HMD | 23 | 3.5857 | 1.8520 | 0.090 |
| ingredient | HMD | 16 | 2.3220 | 2.4268 | |
| Low frequency | Non-HMD | 23 | 69.261 | 15.031 | 0.500 |
| density | HMD | 16 | 72.794 | 16.499 | |
| Low Frequency/ | Non-HMD | 23 | 1.274 | 0.908 | 0.354 |
| High Frequency | HMD | 16 | 1.580 | 1.055 | |
| EEG amplitude (TP) | Non-HMD | 23 | 6.0630 | 0.8989 | 0.833 |
| | HMD | 17 | 6.1124 | 0.5692 | |
| EEG frequency | Non-HMD | 23 | 3.9529 | 3.3702 | 0.800 |
| (MPF) | HMD | 17 | 4.1911 | 2.5434 | |

In the present embodiment, Table 2 shows the comparison of the physiological values of the HMD patients and non-HMD people measured when they are on the mountains.

As shown in Table 2, in the aspect of heart rate and hemoglobin oxygen saturation, the at rest heart rate of the HMD patients is higher than that of the non-HMD people and the hemoglobin oxygen saturation of HMD patients is lower than that of the non-HMD people. In the aspect of respiratory depth and respiratory rate, the respiration total amplitude of the HMD patients is lower than that of the non-HMD people. In the aspect of ECG, both the low frequency density and low frequency/high frequency ingredient of the HMD patients are higher than that of the non-HMD people. And the high frequency ingredient of the HMD patients is lower than that of the non-HMD people, but there is no statistic difference. In the aspect of EEG activity parameters, there is no obvious difference between the EEG amplitude of the HMD patients and that of the non-HMD people.

With reference to FIG. 3A, from the HF and LF % data measured on the flat land, it can be seen that the HF value of the non-HMD people is lower than that of the HMD patients; the LF % value of the non-HMD people is higher than that of the HMD patients.

With reference to FIG. 3B, from the HF and LF % data measured before mountain climbing (under normal pressure and low oxygen condition), it can be seen that the HF value of the non-HMD people is higher than that of the HMD patients; the LF % value of the non-HMD people is higher than that of the HMD patients.

With reference to FIG. 3C, from the HF and LF % data measured on the evening of the first day of mountain climbing, it can be seen that the HF value of the non-HMD people is higher than that of the HMD patients; the LF % value of the non-HMD people is higher than that of the HMD patients.

With reference to FIG. 3D, from the HF and LF % data measured on the evening of the second day of mountain climbing, it can be seen that the HF value of the non-HMD people is lower than that of the HMD patients; the LF % value of the non-HMD people is higher than that of the HMD patients.

With reference to FIG. 4A, from the EEG frequency (mean power frequency, MPF) and EEG amplitude (total power, TP) data measured on the flat land, it can be seen that the EEG frequency value of the non-HMD people is lower than that of the HMD patients; the EEG amplitude value of the non-HMD people is higher than that of the HMD patients.

With reference to FIG. 4B, from the EEG frequency and EEG amplitude data measured before mountain climbing (under normal pressure and low oxygen condition), it can be seen that the EEG frequency value of the non-HMD people is lower than that of the HMD patients; the EEG amplitude value of the non-HMD people is lower than that of the HMD patients.

With reference to FIG. 4C, from the EEG frequency and EEG amplitude data measured on the evening of the first day of mountain climbing, it can be seen that the EEG frequency value of the non-HMD people is higher than that of the HMD patients; the EEG amplitude value of the non-HMD people is higher than that of the HMD patients.

With reference to FIG. 4D, from the EEG frequency and EEG amplitude data measured on the evening of the second day of mountain climbing, it can be seen that the EEG frequency value of the non-HMD people is lower than that of the HMD patients; the EEG amplitude value of the non-HMD people is higher than that of the HMD patients.

Therefore, it can be seen from the statistics of Table 1, Table 2, FIG. 3A to FIG. 3D and FIG. 4A to FIG. 4D, in the aspect of autonomic nerve no matter whether for the non-HMD people or for the HMD patients, the heart rate ingredient on the mountain at rest compares with that on the flat land: the high frequency ingredient is lower; however, the low frequency density and low frequency/high frequency ingredient are higher. Herein, there are noticeable statistic differences among the high frequency ingredient of non-HMD people, the high frequency ingredient of HMD patient, low frequency density and low frequency/high frequency ingredient (p<0.05).

In the aspect of EEG activity parameters, for the non-HMD people, the values at rest measured on the mountain compare with that on the flat land: the EEG amplitude is lower, and the EEG frequency is higher. For the HMD patients, the values at rest measured on the mountain compare with that on the flat land: both the EEG amplitude and frequency are higher. In addition, no matter whether it is for the HMD patients or the non-HMD people, the EEG amplitude thereof on the mountain both are higher than that before mountain climbing, and the differences are noticeable (p<0.05).

In the present embodiment, since everybody customarily wears a watch, if the watch is designed with an ECG analysis device, it will not add additional burden (for example, the carrying weight) to the user; the users are able to watch over their physical condition at any time before or during mountain climbing.

To sum up, the ECG analysis method and the device thereof of the present invention provide the users with their physiological condition before or during mountain climbing, so that preventive treatment can be received. Accordingly, the present invention helps reduce mountain climbers' injury caused by HMD.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. An electrocardiogram (ECG) analysis method diagnosing a subject using non-invasive manner, the ECG analysis method comprising:
measuring the E.C.G. of the subject;
the ECG being transformed to acquire a plurality of heart rate variability (HRV) parameters; and
an analyzed result being output according to the analysis of the HRV parameters.

2. The ECG analysis method of claim 1, wherein these HRV parameters comprise a low frequency density variability parameter and a high frequency variability parameter.

3. The ECG analysis method of claim 2, wherein the nature log calculation is performed to the high frequency variability parameter.

4. The ECG analysis method of claim 2, wherein if the value of the high frequency variability parameter is larger than 5 after the nature log calculation is performed, the analyzed result is that the subject is among the high risk of high mountain disease (HMD).

5. The ECG analysis method of claim 2, wherein if the value of the low frequency density variability parameter is larger than 60 or a specific value, the analyzed result is that the subject is among the high risk of HMD.

6. The ECG analysis method of claim 1, wherein the steps of transforming the ECG to acquire the HRV parameters comprise:
the ECG being digitally transformed, and a plurality of wave peaks of a digital ECG being detected;
each of the wave peaks being counted and confirmed;
a plurality of wave peak intervals of the wave peaks being calculated and acquired;
the wave peaks being calculated to acquire the frequency-domain of the HRV parameters.

7. The ECG analysis method of claim 6, wherein the Fast Fourier Transform is used to perform calculation to the wave peak intervals.

8. An ECG analysis method, comprising:
measuring an EEG of the subject;
performing calculation to the EEG to acquire a plurality of EEG activity parameters; and
analyzing these EEG activity parameters with a plurality of standard EEG activity values in a database and outputting an analyzed result.

9. The ECG analysis method of claim 8, wherein the EEG activity parameters comprise an EEG amplitude and an EEG frequency.

10. The ECG analysis method of claim 9, wherein if the EEG amplitude is lower than the corresponding value in the standard EEG activity values, and the EEG frequencies are higher than the corresponding value in the standard EEG activity values, the analyzed result is that the subject is among the high risk of HMD.

11. An ECG analysis device analyzing the ECG of a subject with a non-invasive method, the analysis device comprising:
a sensing unit, contacting the surface of the arm skin of the subject to measure and output an ECG of the subject;
an analysis unit, comprising a database coupled to the sensing unit, to perform magnifying, filtering, digitizing and a transforming calculation to the ECG; acquiring a plurality of HRV parameters; completing the analysis to the HRV parameters; searching for a corresponding analyzed result in a table in the database; and
an output unit, coupled to the analysis unit to receive and output the analyzed result.

12. The ECG analysis device of claim 11, wherein the HRV parameters comprise a low frequency density variability parameter and a high frequency variability parameter.

13. The ECG analysis device of claim 12, wherein the analysis unit comprises nature log calculation performed to the high frequency variability parameter.

14. The ECG analysis device of claim 12, wherein if the value of the high frequency variability parameter after the nature log calculation is larger than 5, the analyzed result is that the subject is among the high risk of HMD.

15. The ECG analysis device of claim 12, wherein if the value of low frequency density variability parameter is larger than 60 or a specific value, the analyzed result is that the subject is among the high risk of HMD.

16. The ECG analysis device of claim 11, wherein the transforming calculation is Fast Fourier Transform.

17. The ECG analysis device of claim 11, wherein the output unit is a monitor, a printer, a CD burner or a network system.

18. The ECG analysis device of claim 11, wherein the analysis unit is a computer capable of digital signal processing (DSP) to perform frequency-domain analysis, time-domain analysis and non-linear analysis.

19. The ECG analysis device of claim 11 is a watch.

20. An ECG analysis device performing HMD diagnosis to a subject using a non-invasive method, the analysis device comprising:
a sensing unit, contacting the surface of the head of the subject to detect and output an EEG of the subject;
an analysis unit, comprising a database coupled to the sensing unit to perform magnifying, filtering, digitizing and a transforming calculation to the EEG; acquiring a plurality of EEG activity parameters and completing the analysis to EEG activity parameters, searching for a corresponding analyzed result in a table in the database; and
an output unit, coupled to the analysis unit to receive and output the analyzed result.

21. The ECG analysis device of claim 20, wherein the EEG activity parameters comprising an EEG amplitude and an EEG frequency.

22. The ECG analysis device of claim 20, wherein if the EEG amplitude is lower than the corresponding value in the standard EEG activity values, and the E.E.G. frequency is higher than the corresponding value in the standard EEG activity values, the analyzed result is that the subject is among the high risk of HMD.

23. The ECG analysis device of claim 20, wherein the analysis unit is a computer capable of digital signal processing (DSP) to perform frequency-domain analysis, time-domain analysis and non-linear analysis.
